(19) United States
(12) Patent Application Publication
(10) Pub. No.: US 2023/0255266 A1
(43) Pub. Date: Aug. 17, 2023

(54) MAIN BODY, ATOMIZING DEVICE, AND AEROSOL GENERATING DEVICE

(71) Applicant: SHENZHEN WOODY VAPES TECHNOLOGY CO., LTD., Shenzhen, Guangdong (CN)

(72) Inventors: Boyang ZHAO, Shenzhen (CN); Guanyun ZHAO, Shenzhen (CN); Min ZHENG, Shenzhen (CN); Zhiliang ZHANG, Shenzhen (CN)

(73) Assignee: SHENZHEN WOODY VAPES TECHNOLOGY CO., LTD., Shenzhen, Guangdong (CN)

(21) Appl. No.: 18/005,294

(22) PCT Filed: Jun. 23, 2021
(86) PCT No.: PCT/CN2021/101846

§ 371 Date: Jan. 12, 2023

(30) Foreign Application Priority Data
Jan. 30, 2021 (CN) 202110131400.6

(51) Int. Cl. A24F 40/48

(52) U.S. Cl. CPC A24F 40/48

(57) ABSTRACT

A main body, an atomizing device, and an aerosol generating device. The main body includes a housing, a separator, an air inlet passage, and a first electrode. An opening is formed at an end of the housing. The separator is located in the housing and splits an internal space of the housing into a first cavity located on a side away from the opening and a second cavity located on a side of the opening. The air inlet passage is within the first cavity and the separator. The air inlet passage communicates with the outer air through the first cavity, and an air outlet end of the air inlet passage communicates with the second cavity, and protrudes from an end surface of the separator located at the second cavity. The first electrode is at the separator and protrudes from the end surface of the separator located at the second cavity.

The present invention claims the priority of the Chinese Patent Application No. 202110131400.6 filed with the Chinese National Intellectual Property Administration on Jan. 30, 2021, titled “MAIN BODY, ATOMIZING DEVICE, AND AEROSOL GENERATING DEVICE”, which is incorporated by reference herein in its entirety.

### TECHNICAL FIELD

Embodiments of the present invention relate to the field of aerosol technologies, and in particular, to a main body for an aerosol generating device, an atomizing device for an aerosol generating device, and an aerosol generating device.

### BACKGROUND

As hazards of traditional cigarettes are gradually recognized by the public, electronic cigarettes have been increasingly accepted by smoking consumers, with a gradually expanding market share. An electronic cigarette is essentially an aerosol generating device. A main body thereof is connected to an atomizing core. A battery in the main body provides electrical energy to heat a heating element in the atomizing core, atomizing e-liquid in an e-liquid tank to generate aerosol for inhaling by the user.

As an electronic cigarette produces smog by electrically heating and atomizing e-liquid, the electronic cigarette requires an air passage for air circulation to carry the heated smog to an air outlet. However, an air inlet of an electronic cigarette in the conventional art is generally located at a connection between an atomizing device and a main body. As the atomizing device and the main body are provided in a detachable structure, the air inlet is a gap with a large area, which not only makes the air inlet long, but also makes air inlet directions disperse. At this point, air flows cannot be well converged, providing poor airflow stability. As a result, a user needs to use a strong suction force to inhale the air from the air inlet, weakening user experience.

In view of this, an air inlet passage may be provided in the main body to communicate with an air passage structure in the atomizing device, so that air enters through the air inlet passage, instead of entering from the gap between the main body and the atomizing device. However, as an air inlet passage for communicating with the atomizing device is provided in the main body, it is desirable to make improvements accordingly to the structure of the existing connection between the main body and the atomizing device, so as to adapt to the electrical connection and air passage connection between the main body and the atomizing device.

### SUMMARY

In view of this, embodiments of the present invention provide a main body for an aerosol generating device, an atomizing device, and an aerosol generating device, so as to resolve the technical problems to make improvements accordingly to the electrical connection and air passage connection in the conventional art after providing an air inlet passage in the main body.

According to a first aspect of the embodiments of the present invention, a main body for an aerosol generating device is provided, including:

a housing, with an opening formed at one end;

a separator, located in the housing and splitting an internal space of the housing into a first cavity located on a side away from the opening and a second cavity located on a side of the opening;

an air inlet passage, disposed in the first cavity and the separator, where the air inlet passage communicates with the outer air through the first cavity, and an air outlet end of the air inlet passage communicates with the second cavity, and protrudes from an end surface of the separator located at the second cavity; and

a first electrode, disposed at the separator and protruding from the end surface of the separator located at the second cavity.

In a preferred embodiment of the main body for an aerosol generating device according to the embodiments of the present invention, the air outlet end of the air inlet passage is an air pipe disposed in the main body, and an end of the air pipe extends towards the second cavity.

In a preferred embodiment of the main body for an aerosol generating device according to the embodiments of the present invention, the air pipe is a rigid pipe.

In a preferred embodiment of the main body for an aerosol generating device according to the embodiments of the present invention, the air outlet end of the air pipe is flush with an end surface of the first electrode.

In a preferred embodiment of the main body for an aerosol generating device according to the embodiments of the present invention, a length of the air pipe extending into the second cavity is greater than a length of the first electrode extending into the second cavity.

In a preferred embodiment of the main body for an aerosol generating device according to the embodiments of the present invention, both the first electrode and the air pipe are provided in a quantity of two.

In a preferred embodiment of the main body for an aerosol generating device according to the embodiments of the present invention, the two air pipes are centrally symmetrical with respect to the center of the end surface of the separator.

In a preferred embodiment of the main body for an aerosol generating device according to the embodiments of the present invention, the two first electrodes are centrally symmetrical with respect to the center of the end surface of the separator.

In a preferred embodiment of the main body for an aerosol generating device according to the embodiments of the present invention, the first electrode has an elastic needle structure.

In a second aspect of the embodiments of the present invention, an atomizing device for an aerosol generating device is provided, where the atomizing device is configured to couple to the main body provided in the first aspect of the embodiments of the present invention, the air inlet passage is configured to communicate with an air passage structure within the atomizing device, and the first electrode is configured to electrically connect to a second electrode in the atomizing device.

In a preferred embodiment of the atomizing device for an aerosol generating device according to the embodiments of the present invention, the atomizing device has a third cavity for accommodating an air outlet end of the air inlet passage.

In a preferred embodiment of the atomizing device for an aerosol generating device according to the embodiments of the present invention, an air inlet end of the air passage structure is provided at the second electrode.

In a preferred embodiment of the atomizing device for an aerosol generating device according to the embodiments of the present invention, the air inlet end of the air passage structure is separated from the second electrode, and the air inlet end of the air passage structure communicates with the third cavity.

In a preferred embodiment of the atomizing device for an aerosol generating device according to the embodiments of the present invention, the air inlet end of the air passage structure is separated from the second electrode, and the air inlet end of the air passage structure is configured to directly communicate with the air outlet end of the air inlet passage.

In a preferred embodiment of the atomizing device for an aerosol generating device according to the embodiments of the present invention, the atomizing device has a sealing part, where when the atomizing device and the main body are assembled, the sealing part is configured to provide sealing at a connection between the air inlet end of the air passage structure and the air outlet end of the air inlet passage.

According to a third aspect of the embodiments of the present invention, an aerosol generating device is provided, including the main body provided in the first aspect of the embodiments of the present invention and the atomizing device provided in the second aspect of the embodiments of the present invention.

The main body, the atomizing device, and the aerosol generating device provided in the embodiments of the present invention have the following beneficial effects: Air enters into the aerosol generating device through the air inlet passage disposed in the main body, such that the air inlet of the aerosol generating device is no longer dependent on a gap between the atomizing device and the main body, thereby providing better converged and stable air flow through the independent air inlet passage. In addition, the electrical connection between the main body and the atomizing device is implemented through fitting between the first electrode and the second electrode, and the air passage connection between the main body and the atomizing device is implemented through fitting between the air outlet end of the air inlet passage and the air passage structure.

### BRIEF DESCRIPTION OF DRAWINGS

Accompanying drawings constituting a part of the embodiments of the present invention are used to provide a further understanding of the embodiments of the present invention, such that other features, purposes, and advantages of the embodiments of the present invention become more apparent. The schematic accompanying drawings and descriptions thereof of the embodiments of the present invention are used to explain the embodiments of the present invention, and do not constitute an improper limitation on the embodiments of the present invention. In which:

FIG. 1 is a cross-sectional view of a first main body for an aerosol generating device provided in an embodiment of the present invention;

FIG. 2 is a partial enlarged view of part A in FIG. 1;

FIG. 3 is a cross-sectional view of a first atomizing device for an aerosol generating device provided in an embodiment of the present invention;

FIG. 4 is a cross-sectional view of a first aerosol generating device provided in an embodiment of the present invention;

FIG. 5 is a partial enlarged view of part B in FIG. 4;

FIG. 6 is a cross-sectional view of a second main body for an aerosol generating device provided in an embodiment of the present invention;

FIG. 7 is a partial enlarged view of part C in FIG. 6;

FIG. 8 is a cross-sectional view of a second atomizing device for an aerosol generating device provided in an embodiment of the present invention;

FIG. 9 is a cross-sectional view of a second aerosol generating device provided in an embodiment of the present invention;

FIG. 10 is a partial enlarged view of part D in FIG. 9; and

FIG. 11 is a three-dimensional structural diagram of a separator in a main body provided in an embodiment of the present invention.

In the drawings:

100. main body; 200. atomizing device; 300. air inlet passage; 400. air passage structure;

1. atomizing core; 2. mouthpiece; 3. suction hole; 4. power supply; 5. control circuit board; 6. air pipe; 7. first sealing element; 8. first groove; 9. through hole; 10. first cavity; 11. second cavity; 12. housing; 13. separator; 14, second groove; 15. third groove; 16. fourth groove; 17. second sealing element; 18. third sealing element; 19. first electrode; 20. second electrode; 21. air pressure sensor; 22. air pressure sensor mounting bracket; 23. sealing part; 24. air outlet end of the air inlet passage; 25. air inlet end of the air passage structure; 26. third cavity; 27. enclosed space.

### DESCRIPTION OF EMBODIMENTS

To enable those skilled in the art to better understand the solutions of the embodiments of the present invention, the technical solutions in the embodiments of the present invention are described clearly and completely in conjunction with the accompanying drawings in the embodiments of the present invention. Obviously, the described embodiments are only some of the embodiments of the present invention, rather than all the embodiments. Based on the embodiments of the present invention, all other embodiments obtained by those of ordinary skill in the art without creative work should fall within the protection scope of the embodiments of the present invention.

It should be noted that the terms “comprise”/“include”, “have” and any variations thereof used in the description and claims of the embodiments of the present invention and the above-mentioned accompanying drawings are intended to cover non-exclusive inclusions. For example, a system, product, or device including a series of units is not necessarily limited to those clearly listed units, but may include units that are not clearly listed or are inherent to such product or device.

In the embodiments of the present invention, the terms such as “above”, “below”, “inside”, “in”, and “outside” indicate an orientation or positional relationship based on the orientation or positional relationship shown in the accompanying drawings. These terms are mainly intended to better describe the embodiments of the present invention and the examples thereof, and are not intended to limit that the indicated device, element, or component must have a specific orientation, or be constructed and operated in a specific orientation.

In addition, some of the above terms may be used to indicate other meanings in addition to indicating the orientation or positional relationship. For example, the term “above” may also be used to indicate an attachment relationship or a connection relationship in some cases. For those of ordinary skill in the art, specific meanings of these terms in the embodiments of the present invention may be understood as appropriate to specific situations.

In addition, the terms “dispose”, “connect”, and “fasten” should be interpreted broadly. For example, “connect” may indicate a fixed connection, a detachable connection, or an integral structure; or may indicate a mechanical connection or an electrical connection; or may indicate a direct connection, or an indirect connection through an intermediary medium, or an internal connection of two devices, elements, or components. For those of ordinary skill in the art, specific meanings of such terms in the embodiments of the present invention may be understood as appropriate to specific situations.

It should be noted that, without conflict, examples in the embodiments of the present invention and the features in the embodiments may be combined with each other.

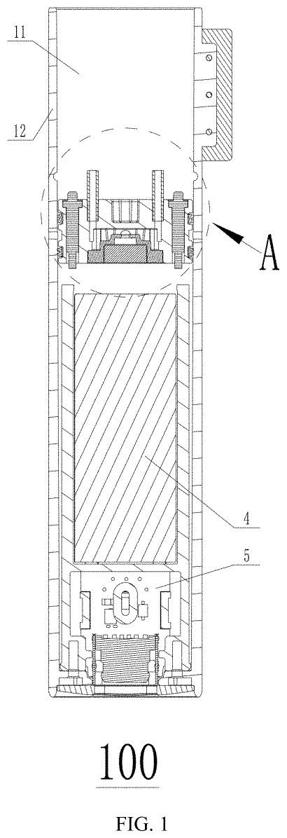

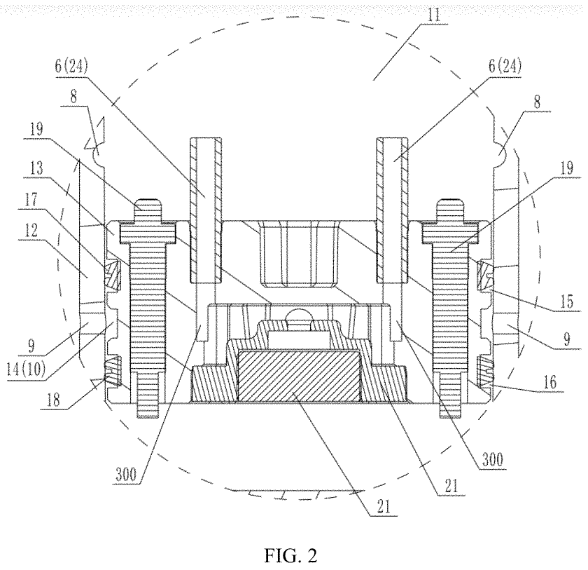

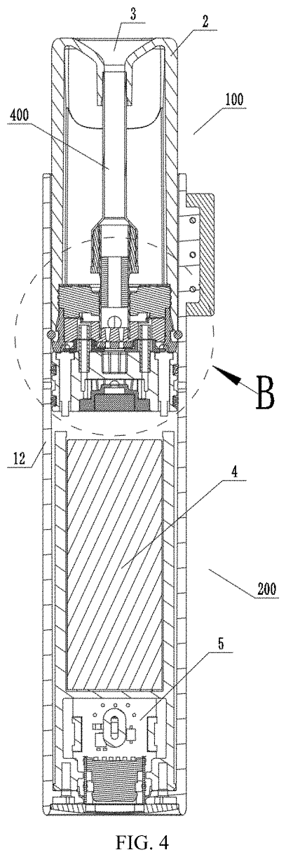

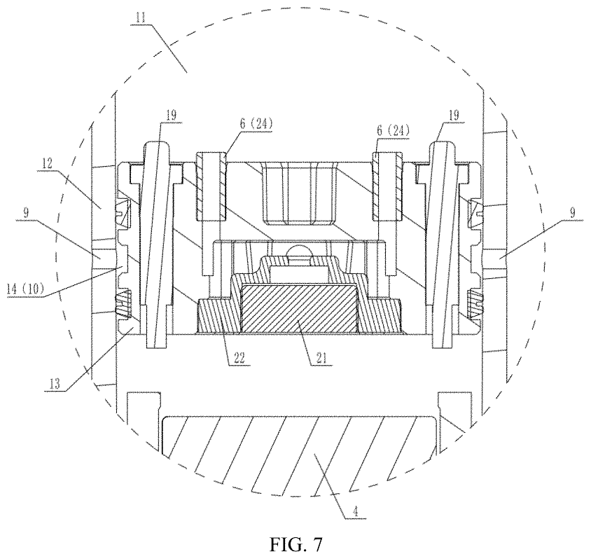

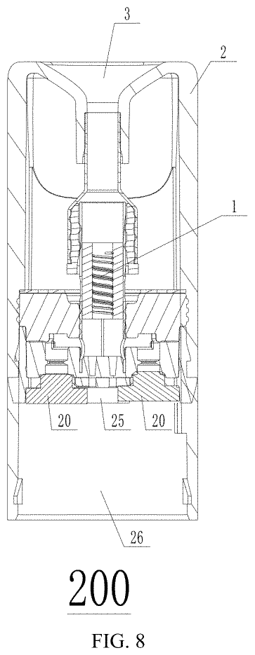

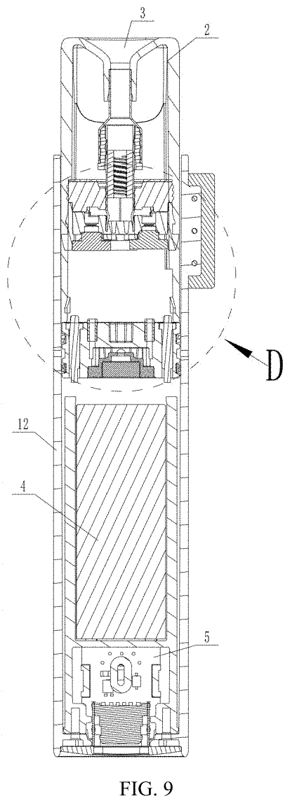

Referring to FIG. 1 to FIG. 11, an aerosol generating device, a main body 100 thereof, and an atomizing device are provided in the embodiments of the present invention. In an embodiment, the aerosol generating device may be in a form such as an electronic cigarette, or certainly may be a medical atomizing device applied in the medical field. The following description uses an aerosol generating device in the form of an electronic cigarette as an example, without imposing any limitation.

The atomizing device 200 for an aerosol generating device is provided with an air passage structure 400. Specifically, the air passage structure 400 of the atomizing device 200 is provided with an atomizing core 1, where the atomizing core 1 is configured to atomize an aerosol matrix (for example, e-liquid and liquid medicine) within the atomizing device 200. When a user is smoking, the external air enters the air passage structure 400 of the atomizing device 200 to carry the aerosol matrix atomized by the atomizing core 1 in the air passage structure 400 to a suction hole 3 at a mouthpiece 2, for inhaling by the user. The atomizing core 1 may be a porous heating generating element, which absorbs the aerosol matrix by capillary force and generates heat to atomize the aerosol matrix. Preferably, the atomizing core 1 may be a porous ceramic heat generating element or the like, which may be further provided with a heat generating film. Certainly, in other embodiments of the present invention, the atomizing core 1 may alternatively be designed as a combination of fiber cotton and a heat generating wire, which is not limited herein.

The main body 100 of the aerosol generating device includes a housing 12 in which a power supply 4 and a control circuit board 5 are provided. The main body 100 is coupled to the atomizing device 200. Specifically, the atomizing device 200 may be detachably connected to the main body 100. Both the power supply 4 in the main body 100 and the atomizing core 1 in the atomizing device 200 are electrically connected to the control circuit board 5 in the main body 100 for supplying power to the atomizing core 1 and controlling the atomizing core 1 to atomize the aerosol matrix when the user inhales, so as to form an vapor for inhaling by the user.

An atomizing device 200 in the conventional art is detachably connected to the main body 100. After the atomizing device 200 is coupled to the main body 100 to form a complete aerosol generating device, a gap may be formed at a connection between the main body 100 and the atomizing device 200. During operation of the aerosol generating device, a path of air from entering to exiting the aerosol generating device is as follows: Air enters the air passage structure 400 of the atomizing device 200 from the gap between the atomizing device 200 and the main body 100; enters the atomizing core 1; then enters the mouthpiece 2 from the atomizing core 1 after combining with the smog atomized by the aerosol matrix such as e-liquid; and finally, gets sucked into the mouth through the suction hole 3 from a cigarette holder (that is, discharged from the aerosol generating device). The air inlet of the whole structure of the aerosol generating device is a gap with a large area, which not only makes the air inlet long, but also makes air inlet directions disperse. At this point, air flows cannot be well converged, providing poor airflow stability. As a result, a user needs to use a strong suction force to inhale the air from the air inlet, weakening user experience.

To resolve the foregoing problems, as shown in FIG. 1 to FIG. 5, the main body 100 for an aerosol generating device according to the embodiments of the present invention includes a separator 13 and an air inlet passage 300. An opening is formed at one end of the housing 12 of the main body 100. The separator 13 is located in the housing 12 and splits an internal space of the housing 12 into a first cavity 10 located on a side away from the opening and a second cavity 11 located on a side of the opening. The air inlet passage 300 is disposed in the first cavity 10 and the separator 13. The air inlet end of the air inlet passage 300 communicates with the outer air, and an air outlet end 24 of the air inlet passage communicates with the second cavity 11. The independent air inlet passage 300 is formed in the structure of the main body 100. The air inlet passage 300 is configured to communicate with the air passage structure 400 in the atomizing device 200. When the main body 100 and the atomizing device 200 are assembled to form an aerosol generating device, the gap between the atomizing device 200 and the main body 100 is allowed to be sealed, such that the air inlet of the aerosol generating device no longer depends on the gap between the atomizing device 200 and the main body 100, and air flow can be well converged and kept stable by using the independent air inlet passage 300. Therefore, the user only needs to use a small suction force to complete the intake, improving the user experience.

To provide an electrical connection between the main body 100 and the atomizing device 200, a first electrode 19 is provided at the separator 13, and the first electrode 19 protrudes from an end surface of the separator 13 at the second cavity 11. The electrode 19 is electrically connected to the control circuit board 5 in the main body 100. Correspondingly, a second electrode 20 is provided on an end surface of the atomizing device 200 that is coupled to the main body 100. The second electrode 20 is electrically connected to the atomizing core 1. When the main body 100 and the atomizing device 200 are assembled, the first electrode 19 is coupled to the second electrode 20, to provide the electrical connection between the control circuit board 5 and the atomizing core 1.

To provide an air passage connection between the main body 100 and the atomizing device 200, the air outlet end 24 of the air inlet passage is an air pipe 6 disposed in the main body 100, and an end of the air pipe 6 extends towards the second cavity 11. The air pipe 6 may be used to isolate the air inlet passage 300 from the assembly gap. As a physical structure, the air pipe 6 may directly or indirectly communicate with the air passage structure 400 in the atomizing device 200, to isolate the air inlet passage 300 from the assembly gap and prevent the outer air from entering through the gap at the connection between the atomizing device 200 and the main body 100, thereby effectively converging air flows, and improving user experience. One end of the air pipe 6 is connected to the separator 13, and the other end extends towards the atomizing device 200, and is configured to communicate with the air passage structure 400. Specifically, the air pipe 6 may be connected to the separator 13 and the air passage structure 400 respectively through plugging. Both the separator 13 and the air inlet end 25 of the air passage structure herein may be made of elastic material, such that the air pipe 6 may be well sealed with the two after being plugged. More preferably, the air pipe 6 may be a rigid pipe made of a material including but not limited to steel, copper, injection plastic, and glass. From the perspective of durability and material availability, the rigid pipe is preferably a steel pipe.

In the foregoing embodiment, the air pipe 6 has a plurality of openings, where one or all of the openings may be disposed at an end of the air pipe 6, to be specific, the axial top of the air pipe 6, for axially introducing or drawing air flows; one or all of the openings may alternatively be disposed on a side wall of the air pipe 6, preferably on a side surface near the axial top of the air pipe 6 for guiding the air flows and for introducing or drawing the air flows radially. The specific arrangement form of the openings is determined according to the arrangement of the air inlet passage 300 and the air passage structure 400, and may be adjusted accordingly by those skilled in the art.

In the foregoing embodiments, a length of the air pipe 6 protruding from the end surface of the separator 13 may be adjusted as required. For example, in some embodiments, the air outlet end of the air pipe 6 is flush with an end surface of the first electrode 19. For another example, in some embodiments, a length of the air pipe 6 extending into the second cavity 11 is greater than a length of the first electrode 19 extending into the second cavity 11.

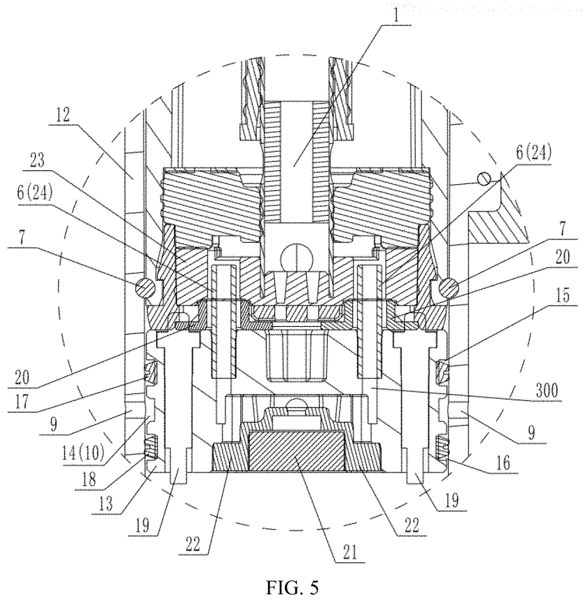

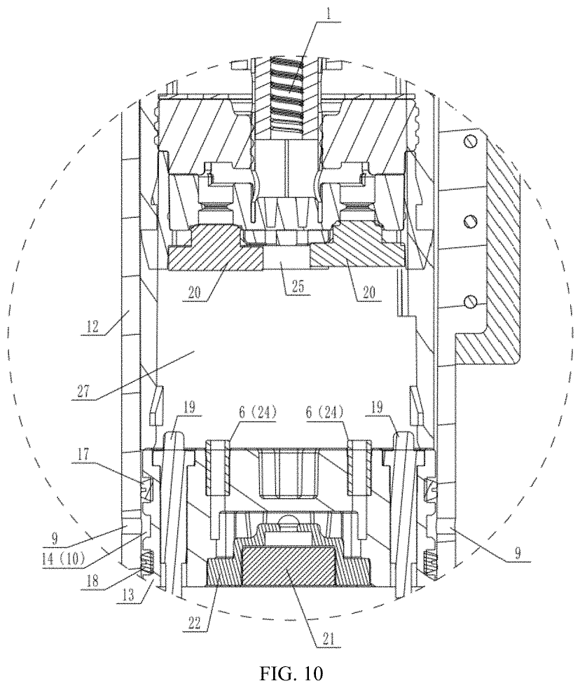

In some embodiments, as shown in FIG. 5 and FIG. 10, the first electrode 19 in the main body 100 and the second electrode 20 in the atomizing device 200 are both provided in a quantity of two. After the main body 100 and the atomizing device 200 are assembled, the two first electrodes 19 and the two second electrodes 20 are respectively coupled.

In some embodiments, as shown in FIG. 1 to FIG. 10, the air inlet end of the air inlet passage 300 is a through hole 9 provided on a side wall of the first cavity 10, and the through hole 9 penetrates through the housing 12, thereby introducing outer air into the air inlet passage 300 through the through hole 9 The air inlet passage 300 of the main body 100 is provided in a quantity of at least one, and the air inlet passage 300 is provided with at least one air inlet end, that is, provided with at least one through hole 9 provided in the housing 12. A plurality of air inlet passages 300 or air inlet ends are provided, which can effectively prevent the aerosol generating device from being unable to operating due to blockage of part of the air inlet passage 300 or the air inlet end caused by misoperation or physical blockage, improving reliability of the product and enhance the user experience. However, to avoid excessive dispersion of air flows, the quantity of the air inlet passage 300 and the air inlet end provided should not be too much. For example, in the accompanying drawings of the embodiments of the present invention, two air inlet passages 300 are provided in the main body 100 of the aerosol generating device. Each of the two air inlet passages 300 is provided with an air inlet end, that is, a through hole 9 provided in a side wall of the housing 12 of the main body 100. The two through holes 9 are symmetrically disposed on two sides of the main body 100, and each of the two air inlet passages 300 is provided with an air outlet end, that is, an air pipe 6 provided in each of the two air inlet passages 300.

On the basis of the foregoing embodiments, the two air pipes 6 are centrally symmetrical with respect to the center of an end face of the separator 13, and the two first electrodes 19 are centrally symmetrical with respect to the center of the end face of the separator 13. Similarly, the two second electrodes 20 are arranged in the atomizing device 200 at positions corresponding to the positions of the two first electrodes 19 respectively.

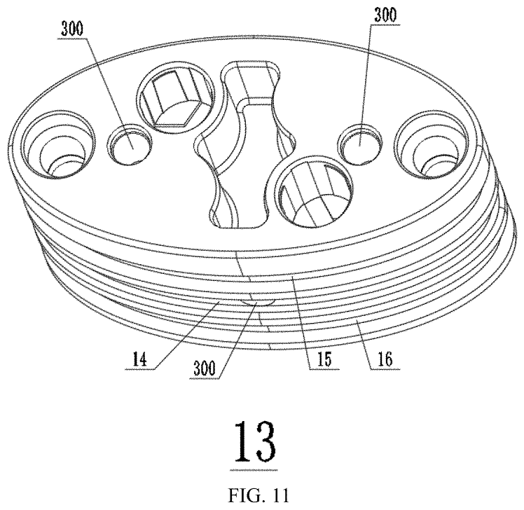

In some embodiments, as shown in FIG. 5, FIG. 10, and FIG. 11, an outer wall of the separator 13 is provided with a second groove 14 in a circumferential direction. The outer wall of the separator 13 fits with an inner wall of the housing 12 of the main body 100. A space defined by the second groove 14 and the inner wall of the housing 12 is a part of the first cavity 10 and is also a part of the air inlet passage 300. The outer air enters the second groove 14 through the through hole 9 in the housing 12, then enters the inside of the separator 13 through the second groove 14, and passes through the separator 13. Designing the first cavity 10 in the form of the second groove 14 may further converge the air flows and improve the user experience.

Optionally, in the foregoing embodiments, to provide sealing of the first cavity 10, the separator 13 may be designed as an elastic structure, such that sealing may be provided between the outer wall of the separator 13 and the inner wall of the housing 12 through interference fit between the separator 13 and the housing 12.

Optionally, in the foregoing embodiments, to provide sealing of the first cavity 10, a third groove 15 and a fourth groove 16 may be provided in the circumferential direction of the outer wall of the separator 13, where the third groove 15 and the fourth groove 16 are respectively located on the upper and lower sides of the second groove 14. A second sealing element 17 is provided in the third groove 15, and a third sealing element 18 is provided in the fourth groove 16. Sealing performance between the separator 13 and the housing 12 may be improved through the second sealing element 17 and the third sealing element 18, where the second sealing element 17 and the third sealing element 18 are preferably sealing rings.

In the foregoing embodiments, the air outlet end 24 of the air inlet passage may communicate with the air passage structure 400 in the atomizing device 200 through various means.

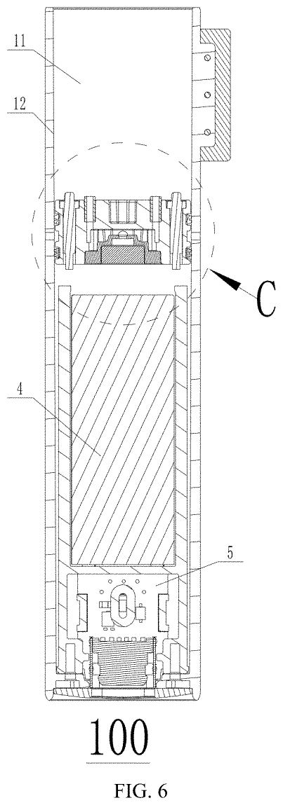

Optionally, as shown in FIG. 6 to FIG. 10, after the atomizing device 200 is assembled with the main body 100 to form an aerosol generating device, the atomizing device 200 is plugged into the first cavity 10 through the opening of the housing 12 of the main body 100. An enclosed space 27 is formed between the atomizing device 200 and the main body 100. The enclosed space 27 may be completely located in the first cavity 10 or partially overlap with the first cavity 10. Both the first electrode 19 and the air outlet end 24 of the air inlet passage extend into the enclosed space 27. A third cavity 26 communicates with the air passage structure 400 of the atomizing device 200. The first electrode 19 is coupled to the second electrode 20. Both the air outlet end 24 of the air inlet passage and the air inlet end 25 of the air passage structure in the atomizing device 200 communicate with the enclosed space 27. The outer air may enter the air passage structure 400 through the air inlet passage 300 and the enclosed space 27 in sequence. This design can avoid the need to make the designed position of the air inlet end 25 of the air passage structure in the atomizing device 200 directly face the air outlet end 24 of the air inlet passage, making the designed positions of the structures in the atomizing device 200 and the main body 100 more free. In this embodiment, the third cavity 26 may be provided at an end in which the atomizing device 200 is coupled to the main body 100. When the atomizing device 200 is engaged with the main body 100, the third cavity 26 and the first cavity 10 together form the foregoing enclosed space 27. The third cavity 26 of the atomizing device 200 may be configured to accommodate the air outlet end 24 of the air inlet passage. It should be noted that the third cavity 26 may only be configured to accommodate the air outlet end 24 of the air inlet passage, with a size and a quantity determined dependent on structural specifications of the actual product, provided that the purpose of the foregoing embodiments of the present invention can be achieved.

The enclosed space 27 needs to avoid communicating with the outer air through the gap between the atomizing device 200 and the main body 100. The inner wall of the second cavity 11 may be formed with the first groove 8 disposed in a circumferential direction and a first sealing element 7 sheathes the outer wall of the atomizing device 200. After an end of the atomizing device 200 is plugged into the second cavity 11 of the main body 100 to form an aerosol generating device, the first sealing element 7 is engaged into the first groove 8 in the inner wall of the second cavity 11 to enhance the sealing effect, where the first sealing element 7 is preferably a sealing ring.

Optionally, after the atomizing device 200 is combined with the main body 100 to assemble an aerosol generating device, when the atomizing device 200 is plugged into the first cavity 10 through the opening of the housing 12 of the main body 100, the first electrode 19 is coupled to the second electrode 20, and the air outlet end 24 of the air inlet passage directly communicate with the air inlet end 25 of the air passage structure in the atomizing device 200. In other words, the air outlet end 24 of the air inlet passage is the air pipe 6, an end of the air pipe 6 may be directly plugged into the air inlet end 25 of the air passage structure in the atomizing device 200, such that the outer air may directly enter the air passage structure 400 through the air inlet passage 300 in sequence, and the air passage is isolated from the assembly gap by using the air pipe 6. For this design, it is necessary to make the designed position of the air inlet end 25 of the air passage structure within the atomizing device 200 directly face the air outlet end 24 of the air inlet passage, to implement the coupling connection therebetween. Although an additional coupling connection structure is added, the connection between the atomizing device 200 and the main body 100 is enhanced through the plugging coupling between the air pipe 6 and the atomizing device 200. In this embodiment, those skilled in the art may determine whether to provide the enclosed space 27 mentioned in the foregoing embodiments according to actual needs.

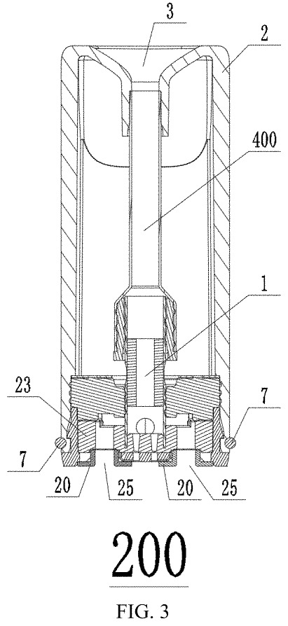

Optionally, as shown in FIG. 3 and FIG. 5, an air inlet end 25 of the air passage structure in the atomizing device 200 is provided at the second electrode 20, where the air inlet end is provided by penetrating through the second electrode 20. As the second electrode 20 may conduct both electric power and air flows, it is not necessary to design the air inlet end 25 of the air passage structure at another position of the atomizing device 200, which may reduce the redundancy in design and assembly, thereby improving simplicity of structural design. In this embodiment, the enclosed space 27 mentioned in the foregoing embodiments may be used to communicate with the air inlet end 25 of the air passage structure, and the air outlet end 24 of the air inlet passage may also directly communicate with the air inlet end 25 of the air passage structure. Those skilled in the art may choose according to design requirements. In the case in which the air outlet end 24 of the air inlet passage directly communicates with the air inlet end 25 of the air passage structure, to avoid interference to the second electrode 20 caused by connection of both the first electrode 19 and the air outlet end 24 of the air inlet passage, the first electrode 19 is preferably in a elastic needle structure, and the second electrode 20 is preferably an electrode nail, where the electrode nail has an inverted T-shaped cross section, with an electrode sheet with a large area, and the air inlet end 25 of the air passage structure penetrates through a middle position of the electrode nail. The first electrode 19 of the elastic needle structure only needs to abut on any position of the electrode nail.

It should be noted that the integral configuration of the air inlet end 25 of the air passage structure and the second electrode 20 is merely a preferred embodiment. In other embodiments, according to specific needs those skilled in the art may still choose schemes of isolation between the air inlet end 25 of the air passage structure and the second electrode 20, communication of the air inlet end 25 of the air passage structure to the air outlet end 24 of the air inlet passage through the enclosed space 27, or direct communication of the air inlet end 25 of air passage structure with the air outlet end 24 of the air inlet passage.

In the foregoing embodiments, in a case where the air outlet end 24 of the air inlet passage in the main body 100 directly communicates with the air inlet end 25 of the air passage structure in the atomizing device 200, the atomizing device 200 is provided with a sealing part 23, where the sealing part 23 is configured to provide sealing between the air inlet end 25 of the air passage structure and the air outlet end 24 of the air inlet passage, when the atomizing device 200 and the main body 100 are assembled. In a case where the second electrode 20 and the air inlet end 25 of the air passage structure are integrally provided, the sealing part 23 may also be used for a sealing connection between the second electrode 20 and other parts of the air passage structure 400. The sealing part 23 is preferably a silicone piece.

In some embodiments, the main body 100 of the aerosol generating device further includes a air pressure sensor 21, and the air pressure sensor 21 is configured to detect the air circulation movement in the air inlet passage 300. The main body 100 is provided with a air pressure sensor mounting bracket 22, where the air pressure sensor mounting bracket 22 is preferably made of silica gel. The air pressure sensor 21 is fixed in the air pressure sensor mounting bracket 22, and the air pressure sensor 21 is electrically connected to the control circuit board 5. Specifically, the power supply 4 may supply power to the atomizing core 1, and an air passage is connected to the air pressure sensor 21. The air pressure sensor 21 may generate changes in electrical parameters varying with negative pressure generated by changes in air flows in the air passage, and output an induction signal to be transmitted to the control circuit board 5. The control circuit board 5 controls power supply for the atomizing core 1 according to the induction signal, for controlling the power supply 4 to supply power to the atomizing core 1, and to atomize the e-liquid when the user smokes through the mouthpiece 2, and to stopping powering the atomizing core 1 when the user stops smoking, such that the atomizing core 1 stops heating. The air inlet passage 300 in the embodiments of the present invention makes the air flows more converged, such that the air pressure sensor 21 senses the air flows with higher sensitivity and accuracy.

It should be noted that other configurations and operations of the main body 100 and the atomizing device 200 for the aerosol generating device provided in the embodiments of the present invention are known to those of ordinary skill in the art, and reference may be made to structures of relevant devices in the conventional art. Therefore, they will not be described in detail herein again.

The embodiments of the present invention also provide an aerosol generating device, including the atomizing device 200 provided in the foregoing embodiments of the present invention and the main body 100 provided in the foregoing embodiments of the present invention. The atomizing device 200 is coupled to the second cavity 11, and the air inlet passage 300 in the main body 100 communicates with the air passage structure 400 in the atomizing device 200. For technical features of the atomizing device 200 and the main body 100, reference may be made to the foregoing description, which will not be repeated herein again. The aerosol generating device disclosed in the embodiments of the present invention includes the main body 100 and the atomizing device 200 provided in the foregoing embodiments, and therefore the aerosol generating device having the main body 100 and the atomizing device 200 also has all the above technical effects, which will not be repeated herein again. Other components and operations of the aerosol generating device are known to those of ordinary skill in the art, and therefore not described in detail herein again.

Some embodiments in this specification are described in a progressive manner. Each embodiment focuses on differences from other embodiments, and same or similar parts between various embodiments can be referred to each other.

The foregoing are only specific implementations of the embodiments of the present invention, such that those skilled in the art may understand or implement the embodiments of the present invention. Various modifications to the embodiments may be apparent to those skilled in the art, and the general principles defined herein may be implemented in other embodiments without departing from the spirit or scope of the embodiments of the present invention. Therefore, the embodiments of the present invention are not limited to the embodiments shown herein, but intended to cover the widest scope consistent with the principles and novel features of the embodiments of the present invention.

1-16. (canceled)

17. A main body for an aerosol generating device, comprising:
a housing, with an opening formed at one end;
a separator, located in the housing and splitting an internal space of the housing into a first cavity located on a side away from the opening and a second cavity located on a side of the opening;
an air inlet passage, disposed in the first cavity and the separator, wherein the air inlet passage communicates with the outer air through the first cavity, and an air outlet end of the air inlet passage communicates with the second cavity, and the separator protrudes and is at an end surface of the second cavity; and
a first electrode, disposed at the separator and the separator protrudes and is at an end surface of the second cavity.

18. The main body according to claim 17, wherein the air outlet end of the air inlet passage is an air pipe disposed in the main body, and an end of the air pipe extends towards the second cavity.

19. The main body according to claim 18, wherein the air pipe is a rigid pipe.

20. The main body according to claim 18, wherein the air outlet end of the air pipe is flush with an end surface of the first electrode.

21. The main body according to claim 18, wherein a length of the air pipe extending into the second cavity is greater than a length of the first electrode extending into the second cavity.

22. The main body according to claim 18, wherein both the first electrode and the air pipe are provided in a quantity of two.

23. The main body according to claim 22, wherein the two air pipes are centrally symmetrical with respect to the center of the end surface of the separator.

24. The main body according to claim 22, wherein the two first electrodes are centrally symmetrical with respect to the center of the end surface of the separator.

25. The main body according to claim 17, wherein the first electrode has an elastic needle structure.

26. An atomizing device for an aerosol generating device, wherein the atomizing device is configured to couple to the main body according to claim 17, the air inlet passage is configured to communicate with an air passage structure within the atomizing device, and the first electrode is configured to electrically connect to a second electrode in the atomizing device.

27. The atomizing device according to claim 26, wherein the atomizing device has a third cavity for accommodating the air outlet end of the air inlet passage.

28. The atomizing device according to claim 26, wherein an air inlet end of the air passage structure is provided at the second electrode.

29. The atomizing device according to claim 27, wherein the air inlet end of the air passage structure is separated from the second electrode, and the air inlet end of the air passage structure communicates with the third cavity.

30. The atomizing device according to claim 26, wherein the air inlet end of the air passage structure is separated from the second electrode, and the air inlet end of the air passage structure is configured to directly communicate with the air outlet end of the air inlet passage.

31. The atomizing device according to claim 30, wherein the atomizing device has a sealing part, and when the atomizing device and the main body are assembled, the sealing part is configured to provide sealing at a connection between the air inlet end of the air passage structure and the air outlet end of the air inlet passage.

32. An aerosol generating device, comprising the main body according to claim 17 and the atomizing device according to claim 26.
